(19) United States
(12) Patent Application Publication
(10) Pub. No.: US 2026/0308106 A1
(43) Pub. Date: Oct. 8, 2026

(54) PROCESS FOR THE ISOMERIZATION OF AROMATIC AMINES

(71) Applicant: BASF SE, Ludwigshafen am Rhein (DE)

(72) Inventors: Trees Maria DE BAERDEMAEKER, Ludwigshafen am Rhein (DE); Matthias NIEDERMAIER, Ludwigshafen am Rhein (DE); Torsten MATTKE, Ludwigshafen am Rhein (DE); Jens FERBITZ, Ludwigshafen am Rhein (DE); Andrei-Nicolae PARVULESCU, Ludwigshafen am Rhein (DE); Dirk DE VOS, Leuven (BE); Ka CHEUNG, Leuven (BE); Sam VAN MINNEBRUGGEN, Leuven (BE); Toshiyuki YOKOI, Tokyo (JP)

(21) Appl. No.: 19/485,332

(22) PCT Filed: May 17, 2024
(86) PCT No.: PCT/EP2024/063668

§ 371 Date: Nov. 18, 2025

(30) Foreign Application Priority Data
May 19, 2023 (EP) 23174260.2

(51) Int. Cl. C07C 209/68

(52) U.S. Cl. CPC C07C 209/68

(57) ABSTRACT

The present invention relates to a process for the isomerization of 2,4′-methylenedianiline and/or, preferably and, 2,2′-methylenedianiline to 4,4′-methylenedianiline, respectively, the process comprising (1) preparing a feed F2 comprising 2,4′-methylenedianiline and/or, preferably and, 2,2′-methylenedianiline and optionally further comprising aniline; (2) providing a catalyst C2 comprising a Bronsted acid; (3) contacting the feed F2 prepared in (1) with the catalyst C2 provided in (2), obtaining a mixture M2 comprising 4,4′-methylenedianiline as a reaction product; (4) optionally separating the 4,4′-methylenedianiline from the reaction product obtained in (3); wherein the feed F2 in (1) comprises 5 wt.-% or less of formaldehyde.

### TECHNICAL FIELD

The present invention relates to a process for the isomerization of 2,4′-methylenedianiline and/or, preferably and, 2,2′-methylenedianiline to 4,4′-methylenedianiline, as well as to a process for the isomerization of 2,2′-methylenedianiline to 2,4′-methylenedianiline.

### INTRODUCTION

Methylene diphenylene diamine and polymethylene polyphenylene polyamines are widely used as intermediates in the production of methylene diphenylene diisocyanates and polymethylene polyphenylene polyisocyanates. The isocyanates then are applied in polymerization reactions with themselves and CH-acidic compounds to yield polyisocyanurates, polyurethanes and polyureas. A mixture of methylenedianiline (MDA) isomers (p,p or 4,4′, o,p or 2,4′, and o,o or 2,2′) and oligomers is produced during this synthesis, wherein 4,4′-MDA is preferred in many applications. This preference is due to the higher reactivity of isocyanate groups in para-position as well as the physical properties resulting from this isomer. While the 2,4′-MDA has values in flexible or soft applications like soft foams or adhesives, the 2,2′-MDA is an undesired side-product.

Further processing of these product streams results in side streams with an increased 2,4′- and/or 2,2′-MDA isomer content. A method for the conversion of these streams into a 4,4′-MDA richer product would be desirable, wherein the conversion of the amino-group in ortho-position of the 2,2′- and/or 2,4′-MDA isomers to the respective isomer with the amino-group in para-position (4,4′-MDA) would be desirable.

P. J. Whitman et al., ‘Protodealkylation of bis(aminophenyl)methanes’, Tetrahedron Letters, Vol. 27, pages 1887-1890, relates to the isomerization of 2,4′-methylenedianiline with Wyoming bentonite in the presence of aniline. EP 0329367 A2 relates to a method of preparing 4,4′-MDA from aniline and a methylenating agent by using a dealuminated Y type zeolite, a fluorine treated dealuminized Y type zeolite or a metal ion-treated product of dealuminized Y type zeolite.

WO 2021/116419 A relates to a catalytic material and to its use for producing one or more of 4,4′-MDA, 2,2′-MDA, 2,4′-MDA or oligomers thereof, wherein the catalytic material comprises an oxidic support, wherein the oxidic support comprises an element Eos1 selected from the group consisting of Ti, Zr, Al, Si, and mixtures of two or more thereof, and further comprising a supported material supported on the oxidic support, wherein the supported material comprises an element EsM1 selected from the group consisting of Ti, Zr, V, Nb, Ta, Mo, W, Ge, Sn, Sc, Y, La, Ce, Nd, Pr, Hf, Cr, Fe, Co, Ni, Cu, Zn, Pb, and mixtures of two or more thereof.

Despite the advances made with regard to the production of 4,4′-MDA, there remains the need for a highly selective process to increase the concentration of 4,4′-MDA, including oligomers and polymers thereof at all stages of the production process by treating side and/or product streams containing or enriched in 2,2′- and/or 2,4′-MDA. In particular, there remains the need for catalysts displaying improved selectivities towards 4,4′-MDA, while suppressing unwanted 2,2′-MDA, including oligomers and polymers thereof, as well as catalysts displaying improved selectivities towards 2,4′-MDA for making use of any unwanted 2,2′-MDA in the feeds to be treated.

### DETAILED DESCRIPTION

Thus, it was the object of the present invention to provide an improved process for the production of 4,4′-methylenedianiline by the isomerization of a feed, preferably of a product feed, comprising one or more of 2,2′-methylenedianiline and 2,4′-methylenedianiline.

Said object is achieved by the process of the present invention, consisting of a highly selective isomerization of 2,4′-methylenedianiline towards 4,4′-methylenedianiline. Surprisingly, it was found that the process of the present invention permits an efficient conversion of a 2,4′-methylenedianiline and/or 2,2′-methylenedianiline comprising product feed to an 4,4′-methylenedianiline enriched product using Bronsted acid and in particular zeolite catalysts with high selectivity towards said isomer. Further, it has unexpectedly been found that the inventive process not only restricts conversion to unwanted side products, in particular 2,2′-methylenedianiline and acridine, but also displays a high selectivity for the isomerization of 2,2′-methylenedianiline to 2,4′-methylenedianiline, the latter being not only valuable as such as a precursor for the production of soft polymers, but also as a precursor for 4,4′-methylenedianiline via the inventive process.

Therefore, the present invention relates to a process for the isomerization of 2,4′-methylenedianiline and/or, preferably and, 2,2′-methylenedianiline to 4,4′-methylenedianiline, respectively, the process comprising
(1) preparing a feed F2 comprising 2,4′-methylenedianiline and/or, preferably and, 2,2′-methylenedianiline and optionally further comprising aniline;
(2) providing a catalyst C2 comprising a Bronsted acid;
(3) contacting the feed F2 prepared in (1) with the catalyst C2 provided in (2), obtaining a mixture M2 comprising 4,4′-methylenedianiline as a reaction product;
(4) optionally separating the 4,4′-methylenedianiline from the reaction product obtained in (3);
   wherein the feed F2 in (1) comprises 5 wt.-% or less of formaldehyde.

It is preferred that the amount of 2,4′-methylenedianiline in the feed F2 in (1) ranges from 1 to 60 wt.-%, preferably 5 to 50 wt.-%, more preferably from 10 to 45 wt.-%, more preferably from 15 to 40 wt.-%, more preferably from 20 to 35 wt.-%.

It is preferred that the feed F2 prepared in (1) and contacted with the catalyst C2 in (3) is in the liquid phase and/or in the gas phase, preferably in the liquid phase. In case that the feed F2 prepared in (1) is in the liquid phase, it is preferred that the liquid hourly space velocity at which the feed F2 obtained in (1) is contacted with the catalyst C2 in (3) is in the range of from 150 to 500 hW.

It is preferred that the concentration of 4,4′-methylenedianiline in the mixture M2 obtained by (3) is greater than the concentration of 4,4′-methylenedianiline which may optionally be comprised in the feed F2 prepared in (1).

It is preferred that the molar ratio of 2,4′-methylenedianiline to 2,2′-methylenedianiline in the feed F2 in (1) is in the range of from 1:1 to 100:1, preferably from 15:1 to 50:1, more preferably from 18:1 to 30:1; more preferably from 20:1 to 28:1.

It is preferred that the feed F2 in (1) comprises aniline, wherein the amount of aniline in the feed F2 in (1) ranges of from 45 to 90 wt.-%, preferably from 50 to 85 wt.-%, more preferably from 55 to 80 wt.-%, more preferably from 60 to 75 wt.-%.

It is preferred that the feed F2 in (1) comprises 5 wt.-% or less formaldehyde, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt.-% or less of formaldehyde, wherein more preferably the feed F2 in (1) is free of formaldehyde.

Further, it is preferred the feed F2 in (1) comprises 5 wt.-% or less of formaldehyde and (C1-C10)alkyl aldehydes, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt-% or less of formaldehyde and (C1-C10)alkyl aldehydes, wherein more preferably the feed F2 in (1) is free of formaldehyde. Yet further, it is preferred that the feed F2 in (1) comprises 5 wt.-% or less of aldehydes, preferably 3 wt.-% or less, more preferably 2 wt.-% or less, more preferably 1 wt.-% or less, more preferably 0.1 wt-% or less, more preferably 0.05 wt.-% or less, more preferably 0.01 wt.-% or less, more preferably 0.005 wt.-% or less, more preferably 0.001 wt-% or less of aldehydes, wherein more preferably the feed F2 in (1) is free of aldehydes.

It is preferred that prior to contacting in (3), the feed F2 in (1) is heated to a temperature T₁, wherein T₁>T_{M}, T_{M} being the melting point of the feed F2, wherein T₁ is preferably in the range of 50 to 220° C., preferably of from 80 to 210° C., preferably of from 100 to 200° C., more preferably from 150 to 190° C., more preferably from 160 to 180° C. It is preferred that the feed F2 in (1) is a feed stream and the reaction in (3) is conducted in a continuous mode.

It is preferred that the feed F2 in (1) further comprises one or more of 4,4′-methylenedianiline, oligomers of 2,2′-methylenedianiline, oligomers of 2,4′-methylenedianiline, and oligomers of 4,4′-methylenedianiline, wherein more preferably the feed F2 in (1) further comprises one or more of oligomers of 2,2′-methylenedianiline, oligomers of 2,4′-methylenedianiline and/or oligomers of 4,4′-methylendianiline.

It is preferred that the feed F2 in (1) is a product feed obtained from a process for the preparation of one or more of 4,4′-methylenedianiline, 2,2′-methylenedianiline, 2,4′-methylenedianiline, and oligomers thereof.

It is preferred that preparing the feed F2 in (1) comprises
(1.1) providing a feed F1a comprising aniline and a feed F1b comprising formaldehyde;
(1.2) contacting the feeds F1a and F1b provided in (1.1), obtaining a mixture M1b;
(1.3) optionally separating water from the mixture M1b obtained in (1.2), obtaining a Mixture M1c;
(1.4) providing a catalyst C1 comprising a Bronsted acid; and
(1.5) contacting the mixture M1b obtained in (1.2) or the mixture M1c obtained in (1.3), preferably the mixture M1c obtained in (1.3), with the catalyst C1 provided in (1.4), obtaining a mixture M1a comprising 2,4′-methylenedianiline.

In case the feed F2 in (1) is prepared from a process comprising steps (1.1) to (1.5), it is preferred that the mixture M1a further comprises one or more of 2,2′-methylenedianiline, 4,4′-methylenedianiline, acridine, and oligomers of 2,2′-methylenedianiline, 4,4′-methylenedianiline, and/or 2,4′-methylenedianiline, preferably one or more of 2,2′-methylenedianiline, 4,4′-methylenedianiline, and oligomers of 2,2′-methylenedianiline, 4,4′-methylenedianiline, and/or 2,4′-methylenedianiline, more preferably one or more of 2,2′-methylenedianiline and 4,4′-methylenedianiline, wherein more preferably the mixture M1a further comprises 2,2′-methylenedianiline.

Yet further, it is preferred that the process further comprises
(1.6) separating 4,4′-methylenedianiline from mixture M1a, obtaining a mixture M1d.

Yet further, it is preferred that the process further comprises
(1.7) separating the catalyst C1 from the mixture M1a obtained in (1.5) or from the mixture M1d obtained in (1.6), preferably by filtration.

Yet further, it is preferred that the process further comprises
(1.8) recycling the catalyst C1 to (1.4).

Yet further, it is preferred that after (1.5), or after (1.5) and prior to (1.6), or after (1.6), or after (1.6) and prior to (1.7), aniline is added to the mixture M1a obtained in (1.5) or to the mixture M1b obtained in (1.6).

Independently thereof, the present invention relates to a process for the isomerization of 2,2′-methylenedianiline to 2,4′-methylenedianiline, the process comprising
(1′) preparing a feed F3 comprising 2,2′-methylenedianiline and optionally further comprising aniline;
(2′) providing a catalyst C3 comprising a Bronsted acid;
(3′) contacting the feed F3 prepared in (1′) with the catalyst C3 provided in (2′), obtaining a mixture M3 comprising 2,4′-methylenedianiline as a reaction product; wherein the feed F3 in (1′) comprises 5 wt.-% or less of formaldehyde.

In case the process comprises steps (1′) to (3′), it is preferred that the process further comprises
(4′) providing a catalyst C2 comprising a Bronsted acid;
(5′) contacting the mixture M3 prepared in (3′) with the catalyst C2 provided in (4′), obtaining a mixture M4 comprising 4,4′-methylenedianiline as a reaction product;
(6′) optionally separating the 4,4′-methylenedianiline from the reaction product obtained in (5′).

It is preferred that independently from each other the Bronsted acid in (2) or (2′) and/or (4′) is a zeolite. In case the Bronsted acid in (2) or (2′) and/or (4′) is a zeolite, it is preferred that the zeolite comprises YO₂ and X₂O₃ in its framework structure, wherein Y stands for a tetravalent element and X stands for a trivalent element. In case the zeolite comprises YO₂ and X₂O₃ in its framework structure, it is preferred that X is selected from the group consisting of Al, B, Ga, and combinations of two or more thereof, wherein X is preferably Al. Further, it is preferred that Y is selected from the group consisting of Si, Ti, Sn, Ge, and combinations of two or more thereof, wherein Y is preferably Si. Further, it is preferred that the zeolite comprises Si and Al in its framework structure, wherein the SiO₂:Al₂O₃ molar ratio of the zeolite ranges from 1 to 45, preferably from 2 to 30, more preferably from 5 to 25, more preferably from 10 to 20, more preferably from 12 to 18. Further, it is preferred that from 95 to 100 weight-% of the zeolite consists of the tetravalent element Y, the trivalent element X, O, and H, preferably from 97 to 100 wt.-%, more preferably from 98 to 100 wt.-%, more preferably from 99 to 100 wt. %, more preferably from 99.5 to 100 wt.-%, more preferably from 99.9 to 100 wt.-%, based on the total weight of the zeolite. Further, it is preferred that from 95 to 100 wt.-% of the zeolite consists of Si, Al, 0, and H, preferably from 97 to 100 wt.-%, more preferably from 98 to 100 wt.-%, more preferably from 99 to 100 wt.-%, more preferably from 99.5 to 100 wt.-%, more preferably from 99.9 to 100 wt.-%, based on the total weight of the zeolite.

In case the process comprises steps (1) to (4) or (4′) to (6′), it is preferred that independently from each other the catalyst C2 in (2) or (4′) further comprises a binder.

In case the process comprises steps (1′) to (3′), it is preferred that the catalyst C3 in (2′) further comprises a binder.

In case the catalyst C2 in (2) or (4′) and/or the catalyst C3 in (2′) comprises a binder, it is preferred that the binder comprises, preferably consists of, one or more selected from the group consisting of titania, zirconia, alumina, silica, silica-alumina, titania-silica, titania-alumina, zirconia-silica, zirconia-alumina, titania-zirconia, and mixtures of two or more thereof, preferably from the group consisting of silica-alumina, titania-silica, titania-alumina, zirconia-silica, zirconia-alumina, titania-zirconia, and mixtures of two or more thereof, wherein more preferably the binder comprises, more preferably consists of, silica-alumina.

In case the process comprises steps (1) to (4) or (4′) to (6′), it is preferred that independently from each other the catalyst C2 in (2) or (4′) is provided as a shaped body, preferably as an extrudate.

In case the process comprises steps (1′) to (3′), it is preferred that the catalyst C3 in (2′) is provided as a shaped body, preferably as an extrudate.

In case the process comprises steps (1) to (4) or (4′) to (6′), it is preferred that independently from each other from 95 to 100 wt.-% of the catalyst C2 provided in (2) or (4′) consists of the zeolite and the optional binder, preferably from 97 to 100 wt.-%, more preferably from 98 to 100 wt.-%, more preferably from 99 to 100 wt.-%, based on the total weight of the catalyst.

In case the process comprises steps (1′) to (3′), it is preferred that from 95 to 100 wt.-% of the catalyst C3 provided in (2′) consists of the zeolite and the optional binder, preferably from 97 to 100 wt.-%, more preferably from 98 to 100 wt.-%, more preferably from 99 to 100 wt.-%, based on the total weight of the catalyst.

In case the process comprises steps (1) to (4) or (4′) to (6′), it is preferred that independently from each other from 95 to 100 wt.-% of the catalyst C2 in (2) or (4′) consists of Si, Al, O, and H, preferably from 97 to 100 wt.-%, more preferably from 98 to 100 wt. %, more preferably from 99 to 100 wt.-%, more preferably from 99.5 to 100 wt.-%, more preferably from 99.9 to 100 wt.-%, based on the total weight of the catalyst.

In case the process comprises step (2′), it is preferred that from 95 to 100 wt.-% of the catalyst C3 in (2′) consists of Si, Al, O, and H, preferably from 97 to 100 wt.-%, more preferably from 98 to 100 wt.-%, more preferably from 99 to 100 wt.-%, more preferably from 99.5 to 100 wt.-%, more preferably from 99.9 to 100 wt.-%, based on the total weight of the catalyst. Further, it is preferred that independently from each other the catalyst C2 in (2) or (4′) is a zeolite comprising 12-membered rings, wherein the number of members in a ring preferably refers to the number of T-atoms in the ring. Further, it is preferred that independently from each other the catalyst C2 in (2) or (4′) is a zeolite having an AFY, BEA, BEC, BOG, CON, FAU, GME, ISV, ITG, IWR, IWS, IWW, LTF, MEI, MOR, MSE, OFF, POS, SAO, SOR, SOV, UOV, UWY, or YFI type framework structure, preferably a BEA, MSE, or YFI structure type or mixtures thereof, more preferably a YFI or MSE structure type, more preferably a MSE structure type. Further, it is preferred that independently from each other the catalyst C2 in (2) or (4′) is a zeolite having an MSE type framework structure, wherein the zeolite is MCM-68 or YNU-2, preferably MCM-68. Further, it is preferred that independently from each other the catalyst C2 in (2) or (4′) is a zeolite having a YFI type framework structure, wherein the zeolite is YNU-5. Further, it is preferred that independently from each other the catalyst C2 in (2) or (4′) is a zeolite having a BEA type framework structure, wherein the zeolite is selected from the group consisting of zeolite beta, [B—Si—O]—BEA, [Ga—Si—O]—BEA, and [Ti—Si—O]—BEA, wherein preferably the preferably the zeolite is zeolite beta. Further, it is preferred that independently from each other the catalyst C2 in (2) or (4′) is a zeolite of which the maximum pore of the zeolite is in the range of from 5 to 12 Å, preferably from 5.5 to 10.5 Å, more preferably from 6 to 10 Å, more preferably from 6.5 to 8.5 Å.

Within the meaning of the present application, the maximum diameter of a sphere which can be included in the framework structure of a zeolite refers to the value obtained by computational geometry by Delaunay triangulation with the following assumptions:
both the framework T- and O-atoms are hard spheres of diameter 2.7 angstrom
all extra-framework atoms (i.e. water, organics and cations) are ignored
the interrupted frameworks are not terminated by hydrogen atoms, i.e. only T and O atoms are considered as hard spheres
the calculations are based on the coordinates of ideal SiO2 frameworks in the highest possible symmetry. Preferably, the values are calculated as described in: “A geometric solution to the largest-free-sphere problem in zeolite frameworks”, M. D. Foster, I. Rivin, M. M. J. Treacy and O. Delgado Friedrichs, Micropor. Mesopor. Mat., 90,32-38, 2006.

In case the process comprises steps (1) to (4) or (4′) to (6′), it is preferred that independently from each other the catalyst C2 in (2) or (4′) is a zeolite of which the maximum diameter of a sphere which can be included in the framework structure of the zeolite is in the range of from 5 to 12 Å, preferably from 5.5 to 10.5 Å, more preferably from 6 to 10 Å, more preferably from 6.5 to 8.5 Å, more preferably from 7 to 8 Å.

In case the process comprises steps (1′) to (3′), it is preferred that the catalyst C3 in (2′) is a zeolite having a BEA, FAU, MSE or MWW type framework structure or mixtures thereof, preferably a BEA, MSE, or MWW structure type, more preferably a MWW structure type. Further, it is preferred that the catalyst C3 in (2′) is a zeolite and the zeolite has an MWW type framework structure, wherein the zeolite is PSH-3, SSZ-25, ERB-1, MCM-22, ITO-1, [Ga—Si—O]-MWW or [Ti—Si—O]-MWW, preferably MCM-22.

It is preferred that independently from each other the Bronsted acid in (2) or (2′) and/or (4′) is an inorganic acid, preferably selected from the group consisting of HCl, HNO₃, H₂SO₄, CF₃SO₃H, and H₃PO₄, including mixtures of two or more thereof, wherein more preferably the Bronsted acid is HCl.

In case the process comprises steps (1) to (4) or (4′) to (6′), it is preferred that independently from each other the catalyst C2 in (2) or (4′) is present in a slurry bed.

In case the process comprises steps (1′) to (3′), it is preferred that the catalyst C3 in (2′) is present in a slurry bed.

It is preferred that independently from each other in (3) or (3′) and/or (5′) the reaction occurs in a fixed bed reactor or in a fluidized bed reactor, preferably in a fixed bed reactor.

It is preferred that independently from each other contacting in (3) or (3′) and/or (5′) is conducted at a temperature in the range of from 130 to 210° C., preferably of from 140 to 200° C., more preferably from 150 to 190° C., more preferably from 160 to 180° C.

It is preferred that independently from each other contacting in (3) or (3′) and/or (5′) is conducted at a pressure in the range of from 0.9 to 1.3 bara.

It is preferred that independently from each other contacting in (3) or (3′) and/or (5′) is conducted for a duration in the range of from 0.1 to 24 h, preferably from 1 to 22 h, preferably from 2 to 20 h, more preferably from 3 to 18 h, more preferably from 5 to 15 h.

In case the process comprises steps (4′) to (6′), it is preferred that the mixture M3 obtained in (3′) and contacted with the catalyst C2 in (5′) is in the liquid phase and/or in the gas phase, preferably in the liquid phase. In case the mixture M3 obtained in (3′) and contacted with the catalyst C2 in (5′) is in the liquid phase, it is preferred that the liquid hourly space velocity at which the mixture M3 obtained in (3′) is contacted with the catalyst C2 in (5′) is in the range of from 150 to 500 hW.

It is preferred that independently from each other contacting in (3) or (3′) and/or (5′) is performed under a gas atmosphere, wherein the gas atmosphere in (3) or (3′) and/or (5′) preferably comprises one or more inert gases, preferably nitrogen and/or argon, more preferably nitrogen.

In case the process comprises steps (1) to (4) or (4′) to (6′), it is preferred that independently from each other the process includes a step of regenerating the catalyst C2 after contacting with the feed in (3) or (5′), wherein the catalyst C2 is preferably regenerated by calcination at a temperature in the range of from 300 to 800° C., preferably from 350 to 700° C., more preferably from 400 to 600° C., more preferably from 450 to 500° C.

In case the process comprises steps (1′) to (3′), it is preferred that the process includes a step of regenerating the catalyst C3 after contacting with the feed in (3′), wherein the catalyst C3 is preferably regenerated by calcination at a temperature in the range of from 300 to 800° C., preferably from 350 to 700° C., more preferably from 400 to 600° C., more preferably from 450 to 500° C.

In case the process includes a step of regenerating the catalyst C2 or C3 after contacting with the feed in (3), (5′) or (3), it is preferred that the calcination is conducted for a period ranging from 1 to 24 h, preferably from 2 to 12 h, more preferably from 3 to 8 h.

In case the process comprises steps (4′) to (6′), it is preferred that the concentration of 4,4′-methylenedianiline in the mixture M4 obtained by (5′) is greater than the concentration of 4,4′-methylenedianiline which may optionally be comprised in the feed F3 prepared in (1′).

In case the process comprises steps (1′) to (3′), it is preferred that the feed F3 in (1′) comprises 2,2′-methylenedianiline and wherein the amount of 2,2′-methylenedianiline in the feed F3 ranges from 1 to 40 wt.-%, preferably 3 to 30 wt.-%, more preferably from 5 to 26 wt.-%, more preferably from 8 to 23 wt.-%, more preferably from 10 to 20 wt.-%.

Further, it is preferred that the feed F3 in (1′) comprises aniline, wherein the amount of aniline in the feed F3 in (1′) ranges of from 45 to 90 wt.-%, preferably from 50 to 85 wt. %, more preferably from 55 to 80 wt.-%, more preferably from 60 to 75 wt.-%. Further, it is preferred that the feed F3 in (1′) comprises 5 wt.-% or less formaldehyde, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt.-% or less of formaldehyde, wherein more preferably the feed F3 in (1′) is free of formaldehyde.

Yet further, it is preferred that the feed F3 in (1′) comprises 5 wt.-% or less of formaldehyde and (C1-C10)alkyl aldehydes, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt-% or less of formaldehyde and (C1-C10)alkyl aldehydes, wherein more preferably the feed F3 in (1′) is free of formaldehyde. Yet further, it is preferred that the feed F3 in (1′) comprises 5 wt.-% or less of aldehydes, preferably 3 wt.-% or less, more preferably 2 wt.-% or less, more preferably 1 wt.-% or less, more preferably 0.1 wt-% or less, more preferably 0.05 wt.-% or less, more preferably 0.01 wt.-% or less, more preferably 0.005 wt.-% or less, more preferably 0.001 wt-% or less of aldehydes, wherein more preferably the feed F3 in (1′) is free of aldehydes. Further, it is preferred that prior to contacting in (3′), the feed F3 in (1′) is heated to a temperature T₂, wherein T₂>T_{M}′, T_{M}′ being the melting point of the feed F3, wherein T₂ is preferably in the range of 50 to 220° C., preferably of from 80 to 210° C., preferably of from 100 to 200° C., more preferably from 150 to 190° C., more preferably from 160 to 180° C. Further, it is preferred that the feed F3 in (1′) is a feed stream and the reaction in (3′) or in (3′) and/or (5′) is conducted in a continuous mode.

It is preferred that the process is conducted as a continuous or as a batch process, preferably as a continuous process.

In case the process comprises steps (1′) to (3′), it is preferred that preparing the feed F3 in (1′) comprises
(1.1′) providing a feed F1a comprising aniline and a feed F1b comprising formaldehyde;
(1.2′) contacting the feeds F1a and F1b provided in (1.1′), obtaining a mixture M1b′;
(1.3′) optionally separating water from the mixture M1b′ obtained in (1.2′), obtaining a mixture M1c′;
(1.4′) providing a catalyst C1 comprising a Bronsted acid; and
(1.5′) contacting the mixture M1b′ obtained in (1.2′) or the mixture M1c′ obtained in
(1.3′), preferably the mixture M1c′ obtained in (1.3′), with the catalyst C1 provided in
(1.4′), obtaining a mixture M1a′ comprising 2,2′-methylenedianiline.

In case the process comprises steps (1.1′) to (1.5′), it is preferred that the mixture M1a′ further comprises one or more of 2,4′-methylenedianiline, 4,4′-methylenedianiline, acridine, and oligomers of 2,2′-methylenedianiline, 4,4′-methylenedianiline, and/or 2,4′-methylenedianiline, preferably one or more of 2,2′-methylenedianiline, 4,4′-methylenedianiline, and oligomers of 2,2′-methylenedianiline, 4,4′-methylenedianiline, and/or 2,4′-methylenedianiline, more preferably one or more of 2,4′-methylenedianiline and 4,4′-methylenedianiline, wherein more preferably the mixture M1a′ further comprises 2,4′-methylenedianiline.

Further, it is preferred that the process further comprises
(1.6′) separating 4,4′-methylenedianiline from mixture M1a′, obtaining a mixture M1d′.

Further, it is preferred that the Bronsted acid in (1.4) or (1.4′) is a solid acid, preferably a zeolite. In case the Bronsted acid in (1.4) or (1.4′) is a solid acid, it is preferred that the catalyst C1 is present in a fixed bed or in a slurry bed. Further, it is preferred that the catalyst C1 is provided as a shaped body, preferably as an extrudate.

In case the process comprises steps (1.1) to (1.5) or (1.1′) to (1.5′), it is preferred that the catalyst C1 is dried prior to (1.1) or (1.1′) in a gas atmosphere, wherein the gas atmosphere has a temperature in the range of from 100 to 300° C., preferably in the range of from 150 to 250° C., more preferably in the range of from 190 to 210° C. Further, it is preferred that the gas atmosphere comprises one or more of nitrogen, oxygen and a mixture thereof, wherein the gas atmosphere is preferably oxygen, air or lean air. Further, it is preferred that the drying is performed for a duration in the range of from 10 to 40 h, preferably in the range of from 20 to 30 h, more preferably in the range of from 23 to 25 h.

In case the process comprises steps (1.1) to (1.5) or (1.1′) to (1.5′), it is preferred that the Bronsted acid in (1.4) or (1.4′) is an inorganic acid, preferably HCl. Further, it is preferred that the catalyst C1 is a heterogeneous or a homogeneous catalyst. Further, it is preferred that the feeds F1a and F1b in (1.1) or (1.1′) are a feed stream and the reaction in (1.5) or (1.5′) is conducted in a continuous mode. Further, it is preferred that the reaction conditions in (1.5) or (1.5′) comprise heating at a temperature in the range of from 100 to 200° C., preferably in the range of from 130 to 170° C., more preferably in the range of from 145 to 155° C. Further, it is preferred that the reaction conditions in (1.5) or (1.5′) comprise a pressure of equal to or smaller than 100 bar, preferably of equal to or smaller than 90, more preferably of equal to or smaller than 70, more preferably of equal to or smaller than 50, more preferably of equal to or smaller than 30, and more preferably of equal to or smaller than 10 bars. Further, it is preferred that (1.5) or (1.5′) is performed under a gas atmosphere, wherein the gas atmosphere in (1.5) or (1.5′) preferably comprises one or more inert gases, preferably nitrogen and/or argon, more preferably nitrogen.

In case the process comprises steps (1.1′) to (1.5′), it is preferred that the process further comprises
(1.7′) separating the catalyst C1 from the mixture M1a′ obtained in (1.5′) or from the mixture M1d′ obtained in (1.6′), preferably by filtration.

Further, it is preferred that the process further comprises
(1.8′) recycling the catalyst C1 to (1.4′).

In case the process comprises steps (1.1) to (1.5) or (1.1′) to (1.5′), it is preferred that after (1.7) and prior to (1.8), or after (1.7′) and prior to (1.8′), the catalyst C1 is not subject to a step of washing or drying, wherein preferably the catalyst C1 is not subject to any treatment after (1.7) and prior to (1.8), or after (1.7′) and prior to (1.8′). Further, it is preferred that separating in (1.6) or (1.6′) is conducted by distillation, preferably by fractional distillation. Further, it is preferred that after (1.5′), or after (1.5′) and prior to (1.6′), or after (1.6′), or after (1.6′) and prior to (1.7′), aniline is added to the mixture M1a′ obtained in (1.5′) or to the mixture M1b′ obtained in (1.6′).

In case the process comprises steps (1) to (4) or (4′) to (6′), it is preferred that separating in (4) or (6′) is conducted by distillation, preferably by fractional distillation.

The present invention is further illustrated by the following set of embodiments and combinations of embodiments resulting from the dependencies and back-references as indicated. In particular, it is noted that in each instance where a range of embodiments is mentioned, for example in the context of a term such as “The process accordingly to any one of embodiments 1 to 4”, every embodiment in this range is meant to be explicitly disclosed for the skilled person i.e. the wording of this term is to be understood by the skilled person as being synonymous to “The process according to any one of embodiments 1, 2, 3 and 4”. Further, it is explicitly noted that the following set of embodiments is not the set of claims determining the extent of protection, but represent a suitably structured part of the description directed to general and preferred aspects of the present invention.
1. A process for the isomerization of 2,4′-methylenedianiline and/or, preferably and, 2,2′-methylenedianiline to 4,4′-methylenedianiline, respectively, the process comprising
   (1) preparing a feed F2 comprising 2,4′-methylenedianiline and/or, preferably and, 2,2′-methylenedianiline and optionally further comprising aniline;
   (2) providing a catalyst C2 comprising a Bronsted acid;
   (3) contacting the feed F2 prepared in (1) with the catalyst C2 provided in (2), obtaining a mixture M2 comprising 4,4′-methylenedianiline as a reaction product;
   (4) optionally separating the 4,4′-methylenedianiline from the reaction product obtained in (3);
   wherein the feed F2 in (1) comprises 5 wt.-% or less of formaldehyde.
2. The process according to embodiment 1, wherein the amount of 2,4′-methylenedianiline in the feed F2 in (1) ranges from 1 to 60 wt.-%, preferably 5 to 50 wt. %, more preferably from 10 to 45 wt.-%, more preferably from 15 to 40 wt.-%, more preferably from 20 to 35 wt.-%.
3. The process according to embodiment 1 or 2, wherein the feed F2 prepared in (1) and contacted with the catalyst C2 in (3) is in the liquid phase and/or in the gas phase, preferably in the liquid phase.
4. The process according to embodiment 3, wherein the liquid hourly space velocity at which the feed F2 obtained in (1) is contacted with the catalyst C2 in (3) is in the range of from 150 to 500 hW.
5. The process according to any one of embodiments 1 to 4, wherein the concentration of 4,4′-methylenedianiline in the mixture M2 obtained by (3) is greater than the concentration of 4,4′-methylenedianiline which may optionally be comprised in the feed F2 prepared in (1).
6. The process according to any one of embodiments 1 to 5, wherein the molar ratio of 2,4′-methylenedianiline to 2,2′-methylenedianiline in the feed F2 in (1) is in the range of from 1:1 to 100:1, preferably from 15:1 to 50:1, more preferably from 18:1 to 30:1; more preferably from 20:1 to 28:1.
7. The process according to any one of embodiments 1 to 6, wherein the feed F2 in (1) comprises aniline, wherein the amount of aniline in the feed F2 in (1) ranges of from 45 to 90 wt.-%, preferably from 50 to 85 wt.-%, more preferably from 55 to 80 wt.-%, more preferably from 60 to 75 wt.-%.
8. The process according to any one of embodiments 1 to 7, wherein the feed F2 in (1) comprises 5 wt.-% or less formaldehyde, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt.-% or less of formaldehyde, wherein more preferably the feed F2 in (1) is free of formaldehyde.
9. The process of embodiment 8, wherein the feed F2 in (1) comprises 5 wt.-% or less of formaldehyde and (C1-C10)alkyl aldehydes, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt-% or less of formaldehyde and (C1-C10)alkyl aldehydes, wherein more preferably the feed F2 in (1) is free of formaldehyde.
10. The process of embodiment 9, wherein the feed F2 in (1) comprises 5 wt.-% or less of aldehydes, preferably 3 wt.-% or less, more preferably 2 wt.-% or less, more preferably 1 wt.-% or less, more preferably 0.1 wt-% or less, more preferably 0.05 wt.-% or less, more preferably 0.01 wt.-% or less, more preferably 0.005 wt.-% or less, more preferably 0.001 wt-% or less of aldehydes, wherein more preferably the feed F2 in (1) is free of aldehydes.
11. The process according to any one of embodiments 1 to 10, wherein prior to contacting in (3), the feed F2 in (1) is heated to a temperature T₁, wherein T₁>T_{M}, T_{M} being the melting point of the feed F2, wherein T₁ is preferably in the range of 50 to 220° C., preferably of from 80 to 210° C., preferably of from 100 to 200° C., more preferably from 150 to 190° C., more preferably from 160 to 180° C.
12. The process according to any one of embodiments 1 to 11, wherein the feed F2 in (1) is a feed stream and the reaction in (3) is conducted in a continuous mode.
13. The process according to any one of embodiments 1 to 12, wherein the feed F2 in (1) further comprises one or more of 4,4′-methylenedianiline, oligomers of 2,2′-methylenedianiline, oligomers of 2,4′-methylenedianiline, and oligomers of 4,4′-methylenedianiline, wherein more preferably the feed F2 in (1) further comprises one or more of oligomers of 2,2′-methylenedianiline, oligomers of 2,4′-methylenedianiline and/or oligomers of 4,4′-methylendianiline.
14. The process according to any one of embodiments 1 to 13, wherein the feed F2 in (1) is a product feed obtained from a process for the preparation of one or more of 4,4′-methylenedianiline, 2,2′-methylenedianiline, 2,4′-methylenedianiline, and oligomers thereof.
15. The process according to any one of embodiments 1 to 14, wherein preparing the feed F2 in (1) comprises
   (1.1) providing a feed F1a comprising aniline and a feed F1b comprising formaldehyde;
   (1.2) contacting the feeds Fla and F1 b provided in (1.1), obtaining a mixture M1b;
   (1.3) optionally separating water from the mixture M1b obtained in (1.2), obtaining a Mixture M1c;
   (1.4) providing a catalyst C1 comprising a Bronsted acid; and
   (1.5) contacting the mixture M1b obtained in (1.2) or the mixture M1c obtained in (1.3), preferably the mixture M1c obtained in (1.3), with the catalyst C1 provided in (1.4), obtaining a mixture M1a comprising 2,4′-methylenedianiline.
16. The process according to embodiment 15, wherein the mixture M1a further comprises one or more of 2,2′-methylenedianiline, 4,4′-methylenedianiline, acridine, and oligomers of 2,2′-methylenedianiline, 4,4′-methylenedianiline, and/or 2,4′-methylenedianiline, preferably one or more of 2,2′-methylenedianiline, 4,4′-methylenedianiline, and oligomers of 2,2′-methylenedianiline, 4,4′-methylenedianiline, and/or 2,4′-methylenedianiline, more preferably one or more of 2,2′-methylenedianiline and 4,4′-methylenedianiline, wherein more preferably the mixture M1a further comprises 2,2′-methylenedianiline.
17. The process according to any one of embodiments 15 or 16, further comprising
   (1.6) separating 4,4′-methylenedianiline from mixture M1a, obtaining a mixture M1d.
18. The process according to any one of embodiments 15 to 17, further comprising
   (1.7) separating the catalyst C1 from the mixture M1a obtained in (1.5) or from the mixture M1d obtained in (1.6), preferably by filtration.
19. The process according to any one of embodiments 15 to 18, further comprising
   (1.8) recycling the catalyst C1 to (1.4).
20. The process according to any one of embodiments 15 to 19, wherein after (1.5), or after (1.5) and prior to (1.6), or after (1.6), or after (1.6) and prior to (1.7), aniline is added to the mixture M1a obtained in (1.5) or to the mixture M1b obtained in (1.6).
21. A process for the isomerization of 2,2′-methylenedianiline to 2,4′-methylenedianiline, the process comprising
   (1′) preparing a feed F3 comprising 2,2′-methylenedianiline and optionally further comprising aniline;
   (2′) providing a catalyst C3 comprising a Bronsted acid;
   (3′) contacting the feed F3 prepared in (1′) with the catalyst C3 provided in (2′), obtaining a mixture M3 comprising 2,4′-methylenedianiline as a reaction product;
   wherein the feed F3 in (1′) comprises 5 wt.-% or less of formaldehyde.
22. The process of embodiment 21, the process further comprising
   (4′) providing a catalyst C2 comprising a Bronsted acid;
   (5′) contacting the mixture M3 prepared in (3′) with the catalyst C2 provided in
   (4′), obtaining a mixture M4 comprising 4,4′-methylenedianiline as a reaction product;
   (6′) optionally separating the 4,4′-methylenedianiline from the reaction product obtained in (5′).
23. The process according to any one of embodiments 1 to 22, wherein independently from each other the Bronsted acid in (2) or (2′) and/or (4′) is a zeolite.
24. The process according to embodiment 23, wherein the zeolite comprises YO₂ and X₂O₃ in its framework structure, wherein Y stands for a tetravalent element and X stands for a trivalent element.
25. The process according to embodiment 24, wherein X is selected from the group consisting of Al, B, Ga, and combinations of two or more thereof, wherein X is preferably Al.
26. The process according to embodiment 24 or 25, wherein Y is selected from the group consisting of Si, Ti, Sn, Ge, and combinations of two or more thereof, wherein Y is preferably Si.
27. The process according to any one of embodiments 23 to 26, wherein the zeolite comprises Si and Al in its framework structure, wherein the SiO₂:Al₂O₃ molar ratio of the zeolite ranges from 1 to 45, preferably from 2 to 30, more preferably from 5 to 25, more preferably from 10 to 20, more preferably from 12 to 18.
28. The process according to any one of embodiments 26 or 27, wherein from 95 to 100 weight-% of the zeolite consists of the tetravalent element Y, the trivalent element X, O, and H, preferably from 97 to 100 wt.-%, more preferably from 98 to 100 wt.-%, more preferably from 99 to 100 wt.-%, more preferably from 99.5 to 100 wt.-%, more preferably from 99.9 to 100 wt.-%, based on the total weight of the zeolite.
29. The process according to any one of embodiments 23 to 28, wherein from 95 to 100 wt.-% of the zeolite consists of Si, Al, 0, and H, preferably from 97 to 100 wt.-%, more preferably from 98 to 100 wt.-%, more preferably from 99 to 100 wt.-%, more preferably from 99.5 to 100 wt.-%, more preferably from 99.9 to 100 wt.-%, based on the total weight of the zeolite.
30. The process according to any one of embodiments 1 to 20 and 22 to 29, wherein independently from each other the catalyst C2 in (2) or (4′) further comprises a binder.
31. The process according to any one of embodiments 21 to 30, wherein the catalyst C3 in (2′) further comprises a binder.
32. The process according to embodiment 30 or 31, wherein the binder comprises, preferably consists of, one or more selected from the group consisting of titania, zirconia, alumina, silica, silica-alumina, titania-silica, titania-alumina, zirconia-silica, zirconia-alumina, titania-zirconia, and mixtures of two or more thereof, preferably from the group consisting of silica-alumina, titania-silica, titania-alumina, zirconia-silica, zirconia-alumina, titania-zirconia, and mixtures of two or more thereof, wherein more preferably the binder comprises, more preferably consists of, silica-alumina.
33. The process according to any one of embodiments 1 to 20 and 22 to 32, wherein independently from each other the catalyst C2 in (2) or (4′) is provided as a shaped body, preferably as an extrudate.
34. The process according to any one of embodiments 21 to 33, wherein the catalyst C3 in (2′) is provided as a shaped body, preferably as an extrudate.
35. The process according to any one of embodiments 1 to 20 and 23 to 34, wherein independently from each other from 95 to 100 wt.-% of the catalyst C2 provided in (2) or (4′) consists of the zeolite and the optional binder, preferably from 97 to 100 wt.-%, more preferably from 98 to 100 wt.-%, more preferably from 99 to 100 wt.-%, based on the total weight of the catalyst.
36. The process according to any one of embodiments 23 to 35, wherein from 95 to 100 wt.-% of the catalyst C3 provided in (2′) consists of the zeolite and the optional binder, preferably from 97 to 100 wt.-%, more preferably from 98 to 100 wt.-%, more preferably from 99 to 100 wt.-%, based on the total weight of the catalyst.
37. The process according to any one of embodiments 1 to 20 and 22 to 36, wherein independently from each other from 95 to 100 wt.-% of the catalyst C2 in (2) or (4′) consists of Si, Al, O, and H, preferably from 97 to 100 wt.-%, more preferably from 98 to 100 wt.-%, more preferably from 99 to 100 wt.-%, more preferably from 99.5 to 100 wt.-%, more preferably from 99.9 to 100 wt.-%, based on the total weight of the catalyst.
38. The process according to any one of embodiments 21 to 37, wherein from 95 to 100 wt.-% of the catalyst C3 in (2′) consists of Si, Al, O, and H, preferably from 97 to 100 wt.-%, more preferably from 98 to 100 wt.-%, more preferably from 99 to 100 wt.-%, more preferably from 99.5 to 100 wt.-%, more preferably from 99.9 to 100 wt.-%, based on the total weight of the catalyst.
39. The process according to any one of embodiments 1 to 20 and 23 to 38, wherein independently from each other the catalyst C2 in (2) or (4′) is a zeolite comprising 12-membered rings, wherein the number of members in a ring preferably refers to the number of T-atoms in the ring.
40. The process according to any one of embodiments 1 to 20 and 23 to 39, wherein independently from each other the catalyst C2 in (2) or (4′) is a zeolite having an AFY, BEA, BEC, BOG, CON, FAU, GME, ISV, ITG, IWR, IWS, IWW, LTF, MEI, MOR, MSE, OFF, POS, SAO, SOR, SOV, UOV, UWY, or YFI type framework structure, preferably a BEA, MSE, or YFI structure type or mixtures thereof, more preferably a YFI or MSE structure type, more preferably a MSE structure type.
41. The process according to any one of embodiments 1 to 20 and 23 to 40, wherein independently from each other the catalyst C2 in (2) or (4′) is a zeolite having an MSE type framework structure, wherein the zeolite is MCM-68 or YNU-2, preferably MCM-68.
42. The process according to any one of embodiments 1 to 20 and 23 to 41, wherein independently from each other the catalyst C2 in (2) or (4′) is a zeolite having a YFI type framework structure, wherein the zeolite is YNU-5.
43. The process according to any one of embodiments 23 to 20 and 23 to 42, wherein independently from each other the catalyst C2 in (2) or (4′) is a zeolite having a BEA type framework structure, wherein the zeolite is selected from the group consisting of zeolite beta, [B—Si—O]—BEA, [Ga—Si—O]-BEA, and [Ti—Si—O]-BEA, wherein preferably the preferably the zeolite is zeolite beta.
44. The process according to any one of embodiments 23 to 20 and 23 to 43, wherein independently from each other the catalyst C2 in (2) or (4′) is a zeolite of which the maximum pore of the zeolite is in the range of from 5 to 12 Å, preferably from 5.5 to 10.5 Å, more preferably from 6 to 10 Å, more preferably from 6.5 to 8.5 Å.
45. The process according to any one of embodiments 1 to 20 and 23 to 44, wherein independently from each other the catalyst C2 in (2) or (4′) is a zeolite of which the maximum diameter of a sphere which can be included in the framework structure of the zeolite is in the range of from 5 to 12 Å, preferably from 5.5 to 10.5 Å, more preferably from 6 to 10 Å, more preferably from 6.5 to 8.5 Å, more preferably from 7 to 8 Å.
46. The process according to any one of embodiments 23 to 45, wherein the catalyst C3 in (2′) is a zeolite having a BEA, FAU, MSE or MWW type framework structure or mixtures thereof, preferably a BEA, MSE, or MWW structure type, more preferably a MWW structure type.
47. The process according to any one of embodiments 23 to 46, wherein the catalyst C3 in (2′) is a zeolite and the zeolite has an MWW type framework structure, wherein the zeolite is PSH-3, SSZ-25, ERB-1, MCM-22, ITQ-1, [Ga—Si—O]-MWW or [Ti—Si—O]-MWW, preferably MCM-22.
48. The process according to any one of embodiments 1 to 47, wherein independently from each other the Bronsted acid in (2) or (2′) and/or (4′) is an inorganic acid, preferably selected from the group consisting of HCl, HNO₃, H₂SO₄, CF₃SO₃H, and H₃PO₄, including mixtures of two or more thereof, wherein more preferably the Bronsted acid is HCl.
49. The process according to embodiment 48, wherein independently from each other the catalyst C2 in (2) or (4′) is present in a slurry bed.
50. The process according to embodiment 48 or 49, wherein the catalyst C3 in (2′) is present in a slurry bed.
51. The process according to any one of embodiments 1 to 50, wherein independently from each other in (3) or (3′) and/or (5′) the reaction occurs in a fixed bed reactor or in a fluidized bed reactor, preferably in a fixed bed reactor.
52. The process according to any one of embodiments 1 to 51, wherein independently from each other contacting in (3) or (3′) and/or (5′) is conducted at a temperature in the range of from 130 to 210° C., preferably of from 140 to 200° C., more preferably from 150 to 190° C., more preferably from 160 to 180° C.
53. The process according to any one of embodiments 1 to 52, wherein independently from each other contacting in (3) or (3′) and/or (5′) is conducted at a pressure in the range of from 0.9 to 1.3 bara.
54. The process according to any one of embodiments 1 to 53, wherein independently from each other contacting in (3) or (3′) and/or (5′) is conducted for a duration in the range of from 0.1 to 24 h, preferably from 1 to 22 h, preferably from 2 to 20 h, more preferably from 3 to 18 h, more preferably from 5 to 15 h.
55. The process according to any one of embodiments 22 to 54, wherein the mixture M3 obtained in (3′) and contacted with the catalyst C2 in (5′) is in the liquid phase and/or in the gas phase, preferably in the liquid phase.
56. The process according to embodiment 55, wherein the liquid hourly space velocity at which the mixture M3 obtained in (3′) is contacted with the catalyst C2 in (5′) is in the range of from 150 to 500 hW.
57. The process according to any one of embodiments 1 to 56, wherein independently from each other contacting in (3) or (3′) and/or (5′) is performed under a gas atmosphere, wherein the gas atmosphere in (3) or (3′) and/or (5′) preferably comprises one or more inert gases, preferably nitrogen and/or argon, more preferably nitrogen.
58. The process according to any one of embodiments 1 to 20 and 22 to 57, wherein independently from each other the process includes a step of regenerating the catalyst C2 after contacting with the feed in (3) or (5′), wherein the catalyst C2 is preferably regenerated by calcination at a temperature in the range of from 300 to 800° C., preferably from 350 to 700° C., more preferably from 400 to 600° C., more preferably from 450 to 500° C.
59. The process according to any one of embodiments 21 to 58, wherein the process includes a step of regenerating the catalyst C3 after contacting with the feed in (3′), wherein the catalyst C3 is preferably regenerated by calcination at a temperature in the range of from 300 to 800° C., preferably from 350 to 700° C., more preferably from 400 to 600° C., more preferably from 450 to 500° C.
60. The process according to embodiment 58 or 59, wherein the calcination is conducted for a period ranging from 1 to 24 h, preferably from 2 to 12 h, more preferably from 3 to 8 h.
61. The process according to any one of embodiments 22 to 60, wherein the concentration of 4,4′-methylenedianiline in the mixture M4 obtained by (5′) is greater than the concentration of 4,4′-methylenedianiline which may optionally be comprised in the feed F3 prepared in (1′).
62. The process according to any one of embodiments 21 to 61, wherein the feed F3 in (1′) comprises 2,2′-methylenedianiline and wherein the amount of 2,2′-methylenedianiline in the feed F3 ranges from 1 to 40 wt.-%, preferably 3 to 30 wt.-%, more preferably from 5 to 26 wt.-%, more preferably from 8 to 23 wt.-%, more preferably from 10 to 20 wt.-%.
63. The process according to any one of embodiments 21 to 62, wherein the feed F3 in (1′) comprises aniline, wherein the amount of aniline in the feed F3 in (1′) ranges of from 45 to 90 wt.-%, preferably from 50 to 85 wt.-%, more preferably from 55 to 80 wt.-%, more preferably from 60 to 75 wt.-%.
64. The process according to any one of embodiments 21 to 63, wherein the feed F3 in (1′) comprises 5 wt.-% or less formaldehyde, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt.-% or less of formaldehyde, wherein more preferably the feed F3 in (1′) is free of formaldehyde.
65. The process of embodiment 64, wherein the feed F3 in (1′) comprises 5 wt.-% or less of formaldehyde and (C1-C10)alkyl aldehydes, preferably 3 wt.-% or less, more preferably 2 wt.-% or less, more preferably 1 wt.-% or less, more preferably 0.1 wt.-% or less, more preferably 0.05 wt.-% or less, more preferably 0.01 wt.-% or less, more preferably 0.005 wt.-% or less, more preferably 0.001 wt-% or less of formaldehyde and (C1-C10)alkyl aldehydes, wherein more preferably the feed F3 in (1′) is free of formaldehyde.
66. The process of embodiment 65, wherein the feed F3 in (1′) comprises 5 wt.-% or less of aldehydes, preferably 3 wt.-% or less, more preferably 2 wt.-% or less, more preferably 1 wt.-% or less, more preferably 0.1 wt-% or less, more preferably 0.05 wt.-% or less, more preferably 0.01 wt.-% or less, more preferably 0.005 wt.-% or less, more preferably 0.001 wt-% or less of aldehydes, wherein more preferably the feed F3 in (1′) is free of aldehydes.
67. The process according to any one of embodiments 21 to 66, wherein prior to contacting in (3′), the feed F3 in (1′) is heated to a temperature T₂, wherein T₂>T_{M}′, T_{M}′ being the melting point of the feed F3, wherein T₂ is preferably in the range of 50 to 220° C., preferably of from 80 to 210° C., preferably of from 100 to 200° C., more preferably from 150 to 190° C., more preferably from 160 to 180° C.
68. The process according to any one of embodiments 21 to 67, wherein the feed F3 in (1′) is a feed stream and the reaction in (3′) or in (3′) and/or (5′) is conducted in a continuous mode.
69. The process according to any one of embodiments 1 to 68, wherein the process is conducted as a continuous or as a batch process, preferably as a continuous process.
70. The process according to any one of embodiments 21 to 69, wherein preparing the feed F3 in (1′) comprises
   (1.1′) providing a feed F1a comprising aniline and a feed F1b comprising formaldehyde;
   (1.2′) contacting the feeds Fla and F1b provided in (1.1′), obtaining a mixture M1 b′
   (1.3′) optionally separating water from the mixture M1b′ obtained in (1.2′), obtaining a mixture M1c′;
   (1.4′) providing a catalyst C1 comprising a Bronsted acid; and
   (1.5′) contacting the mixture M1b′ obtained in (1.2′) or the mixture M1c′ obtained in (1.3′), preferably the mixture M1c′ obtained in (1.3′), with the catalyst C1 provided in (1.4′), obtaining a mixture M1a′ comprising 2,2′-methylenedianiline.
71. The process according to embodiment 70, wherein the mixture M1a′ further comprises one or more of 2,4′-methylenedianiline, 4,4′-methylenedianiline, acridine, and oligomers of 2,2′-methylenedianiline, 4,4′-methylenedianiline, and/or 2,4′-methylenedianiline, preferably one or more of 2,2′-methylenedianiline, 4,4′-methylenedianiline, and oligomers of 2,2′-methylenedianiline, 4,4′-methylenedianiline, and/or 2,4′-methylenedianiline, more preferably one or more of 2,4′-methylenedianiline and 4,4′-methylenedianiline, wherein more preferably the mixture M1a′ further comprises 2,4′-methylenedianiline.
72. The process according to any one of embodiments 70 or 71, further comprising
   (1.6′) separating 4,4′-methylenedianiline from mixture M1a′, obtaining a mixture M1d′.
73. The process according to any one of embodiments 70 to 72, wherein the Bronsted acid in (1.4) or (1.4′) is a solid acid, preferably a zeolite.
74. The process according to any one of embodiments 15 to 20 and 70 to 73, wherein the catalyst C1 is present in a fixed bed or in a slurry bed.
75. The process according to any one of embodiments 15 to 20 and 70 to 74, wherein the catalyst C1 is provided as a shaped body, preferably as an extrudate.
76. The process according to any one of embodiments 15 to 20 and 70 to 75, wherein the catalyst C1 is dried prior to (1.1) or (1.1′) in a gas atmosphere, wherein the gas atmosphere has a temperature in the range of from 100 to 300° C., preferably in the range of from 150 to 250° C., more preferably in the range of from 190 to 210° C.
77. The process according to embodiment 76, wherein the gas atmosphere comprises one or more of nitrogen, oxygen and a mixture thereof, wherein the gas atmosphere is preferably oxygen, air or lean air.
78. The process according to any one of embodiments 76 or 77, wherein the drying is performed for a duration in the range of from 10 to 40 h, preferably in the range of from 20 to 30 h, more preferably in the range of from 23 to 25 h.
79. The process according to any one of embodiments 15 to 20 and 70 to 78, wherein the Bronsted acid in (1.4) or (1.4′) is an inorganic acid, preferably HCl.
80. The process according to any one of embodiments 15 to 20 and 70 to 79, wherein the catalyst C1 is a heterogeneous or a homogeneous catalyst.
81. The process according to any one of embodiments 15 to 20 and 70 to 80, wherein the feeds F1a and F1b in (1.1) or (1.1′) are a feed stream and the reaction in (1.5) or (1.5′) is conducted in a continuous mode.
82. The process according to any one of embodiments 15 to 20 and 70 to 81, wherein the reaction conditions in (1.5) or (1.5′) comprise heating at a temperature in the range of from 100 to 200° C., preferably in the range of from 130 to 170° C., more preferably in the range of from 145 to 155° C.
83. The process according to any one of embodiments 15 to 20 and 70 to 82, wherein the reaction conditions in (1.5) or (1.5′) comprise a pressure of equal to or smaller than 100 bar, preferably of equal to or smaller than 90, more preferably of equal to or smaller than 70, more preferably of equal to or smaller than 50, more preferably of equal to or smaller than 30, and more preferably of equal to or smaller than 10 bars.
84. The process according to any one of embodiments 15 to 20 and 70 to 83, wherein (1.5) or (1.5′) is performed under a gas atmosphere, wherein the gas atmosphere in (1.5) or (1.5′) preferably comprises one or more inert gases, preferably nitrogen and/or argon, more preferably nitrogen.
85. The process according to any one of embodiments 70 to 84, further comprising
(1.7′) separating the catalyst C1 from the mixture M1a′ obtained in (1.5′) or from the mixture M1d′ obtained in (1.6′), preferably by filtration.
86. The process according to any one of embodiments 70 to 85, further comprising
(1.8′) recycling the catalyst C1 to (1.4′).
87. The process according to embodiment 15 to 20 and 70 to 86, wherein after (1.7) and prior to (1.8), or after (1.7′) and prior to (1.8′), the catalyst C1 is not subject to a step of washing or drying, wherein preferably the catalyst C1 is not subject to any treatment after (1.7) and prior to (1.8), or after (1.7′) and prior to (1.8′).
88. The process according to any of embodiments 15 to 20 and 70 to 87, wherein separating in (1.6) or (1.6′) is conducted by distillation, preferably by fractional distillation.
89. The process according to any one of embodiments 15 to 20 and 70 to 88, wherein after (1.5′), or after (1.5′) and prior to (1.6′), or after (1.6′), or after (1.6′) and prior to (1.7′), aniline is added to the mixture M1a′ obtained in (1.5′) or to the mixture M1b′ obtained in (1.6′).
90. The process according to any one of embodiments 1 to 20 and 22 to 89, wherein separating in (4) or (6′) is conducted by distillation, preferably by fractional distillation.

### EXAMPLES

### Reference Example 1: Determination of Si/Al Content

The determination of the Si/Al ratio of the synthesized materials was done using inductively coupled plasma-optical emission spectroscopy (ICP-OES). This was done using a Varian 720-ES with a double-pass glass cyclonic spray chamber with a SeaSpray concentric glass nebulizer and a high solids torch.

For sample digestion, 100 mg of sample and 500 mg LiBO₂ were homogenized and added to a graphite crucible. This crucible was then heated in an oven to 1000° C. for 15 min. Afterwards, the crucible was emptied in a Teflon beaker containing 50 mL HNO₃ (0.42 N). Finally, the graphite precipitant was removed and the sample was diluted accordingly. Quantification of the Si and Al content was done by using a calibration curve for both elements.

### Reference Example 2: Kinetic Model for MDA Isomerization

To determine apparent rate constants associated with the isomerization of methylenedianilines, a kinetic model was constructed. Herein following assumptions were made. First of all, the isomerization reaction was considered to be a first order reaction in the corresponding MDA concentration. The oligomerization reaction was described as a second order reaction in the total concentration of MDA ([2,2′]+[2,4′]+[4,4′]). Furthermore, the oligomerization reaction was assumed to be irreversible. As was shown in mechanistic studies, the isomerization can follow either a mono- or bimolecular pathway. Since aniline affected these rate constants significantly, an extra term was added to the rate constants to take into account the role of aniline.

### Example 1: Effect of Aniline Addition on the Catalytic Process

First, dry beta zeolite (100 mg), 2,4′-methylenedianiline (500 mg) and x mL aniline, wherein x ranges of from 0 to 1.2 mL, were added to a GC vial. The GC vial was flushed with Ar and the vial was heated to 170° C. After reaction, THF was added to dilute/dissolve the reaction mixture. The obtained products were identified by GC-MS. For quantification, tetradecane was added as an external standard and analysis was done using GC. Rate constants were calculated according to Reference Example 2.

TABLE 1
Effect of aniline addition on MDA isomerization kinetics.

| | Aniline | Aniline | k_{iso, 1} (4,4′) | k_{iso, -1} | k_{iso, 2} (2,2′) | k_{oligo} |
|---|---|---|---|---|---|---|
| | (mL) | (M) | (10⁻⁶ s⁻¹)^{c} | (10⁻⁶ s⁻¹)^{c} | (10⁻⁶ s⁻¹)^{c} | (10⁻⁶ s⁻¹)^{c} |
| Example 2 | 1.2 | 7.7 | 5.3 | 0.5 | 0.3 | 0.2 |
| Example 3 | 0.8 | 6.7 | 6 | 20 | 0.7 | 0.1 |
| Example 4 | 0.6 | 5.9 | 8.4 | 19 | 0.5 | 0.3 |
| Example 5 | 0.4 | 4.8 | 9.6 | 40 | 0.9 | 0.3 |
| Example 6 | 0.2 | 3.1 | 28 | 100 | 1.2 | 1.6 |
| Example 7 | 0 | 0 | 3.8 | 8.0 | 1.5 | 2.4 |

^{c}The kinetic values represent respectively the following: k_{iso, 1} (2,4′→ 4,4′), k_{iso, -1} (4,4′→ 2,4′), k_{iso, 2} (2,4′→ 2,2′) and k_{oligo} (MDA→ oligomer)

The effect of aniline addition on the kinetic parameters was evaluated first (see FIG. 2). Aniline affects both k_{iso,2} and k_{oligo} especially the formation of 2,2′-MDA and oligomers in a negative fashion over the entire concentration range. In contrast, the formation of 4,4′-MDA (k_{iso,1}) is initially strongly enhanced upon addition of aniline; only from high aniline concentrations onwards, the rate constant diminishes.

### Example 8: Catalytic Testing

First, a catalyst (100 mg, see table 2), 2,4′-MDA (500 mg) and 1.2 mL aniline were added to a GC vial. The GC vial was flushed with Ar and the vial was heated to 170° C.

After the reaction, THF was added to dilute the reaction mixture. The obtained products were identified by GC-MS. For quantification, tetradecane was added as an external standard and analysis was done using GC. Rate constants were calculated according to Reference Example 2.

TABLE 2
Results from catalytic testing with 2,4′-MDA as starting material.

| | | | | k_{iso, 1} | | |
|---|---|---|---|---|---|---|
| | | | | (4,4′) | k_{iso, 2} (2,2′) | k_{oligo} |
| | Catalyst | Topology | Si/Al | (10⁻⁶ s⁻¹)^{c} | (10⁻⁶ s⁻¹)^{c} | (10⁻⁷ s⁻¹)^{c} |
| Example 9 | HCl^{a} | — | | 1.9 | 0.2 | 11.4 |
| Example 10 | CF₃SO₃H^{b} | — | | 2.7 | 1.0 | 4.2 |
| Example 11 | ZSM-5 | MFI | 15 | 0.4 | 0.2 | 0.4 |
| Example 12 | mordenite | MOR | 10 | 0.6 | 0.3 | 0.4 |
| Example 13 | zeolite beta | BEA | 12.5 | 5.3 | 0.25 | 2.3 |
| Example 14 | nanobeta | BEA | 23 | 3.3 | 2.5 | 0.02 |
| Example 15 | faujasite | FAU | 40 | 1.9 | 0.3 | 0.1 |
| Example 16 | MCM-68 | MSE | 10.8 | 4.7 | 0.2 | 0.2 |
| Example 17 | UZM-35 | MSE | 7.8 | 1.0 | 0.3 | 0.5 |
| Example 18 | YNU-5 | YFI | 9.8 | 4.8 | 0.6 | 0.2 |
| Example 19 | MCM-22 | MWW | 14 | 0.5 | 19.9 | 2.0 |

^{a}In case of HCl as catalyst: 50 μL of a 37 wt.-% solution of HCl was used.
^{b}In case of CF₃SO₃H as catalyst: 50 μL CF₃SO₃H was used.
^{c}The kinetic values represent respectively the following: k_{iso, 1} (2,4′→ 4,4′), k_{iso, 2} (2,4′→ 2,2′), and k_{oligo} (MDA→ oligomer).

### Isomerization of 2,4′-MDA to 4,4′-MDA

Compared to the reference case (i.e. with CF₃SO₃H as catalyst, see FIG. 4), all zeolites suppressed oligomerization, indicating that the confined environment surrounding the acid site inhibits formation of bulky products (see FIG. 3). Zeolites with MFI (ZSM-5, see FIG. 5) or MOR (Mordenite) topologies were found to be inactive, likely due to pore blocking. Also FAU yielded moderate isomerization rates, although possessing the most spacious pores. Beta gave the highest isomerization rates towards the preferred 4,4′-MDA (see FIG. 7).

On the other hand, MCM-68 and YNU-5 (see FIGS. 8 and 9 respectively) also yielded promising results, especially in the suppression of 2,2′-MDA (k_{iso,2}) and oligomers (k_{oligo}). For the MSE topology, the synthesis route affected its activity as MCM-68 was found to be more active than UZM-35. Finally, MCM-22 yielded peculiar results, giving a strong, though undesired selectivity towards 2,2′-MDA. The zeolite catalysts were also found to be easily recyclable via calcination.

### Isomerization of 2,2′-MDA to 2,4′-MDA

First, a catalyst (50 mg, see table 2), 2,2′-MDA (250 mg) and 0.3 mL aniline were added to a GC vial. The GC vial was flushed with Ar and the vial was heated to temperature T, wherein T is 170, 200 or 220° C. (see table 3). After the reaction, THF was added to dilute the reaction mixture. The obtained products were identified by GC-MS. For quantification, tetradecane was added as an external standard and analysis was done using GC. Rate constants were calculated according to Reference Example 2.

TABLE 3
Results from catalytic testing with 2,2′-MDA as starting material

| | | T | k_{iso,-2} | k_{acr} | k_{iso,-2}/ |
|---|---|---|---|---|---|
| Experiment | Catalyst | (° C.) | (10⁻⁵ s⁻¹) | (10⁻⁵ s⁻¹) | k_{acr} |
| Example 20 | zeolite beta | 200 | 1.1 | 0.8 | 1.375 |
| Example 21 | zeolite beta | 220 | 2.5 | 2.4 | 1.04 |
| Example 22 | MCM-68 | 220 | 5.0 | 1.9 | 2.63 |
| Example 23 | MCM-22 | 200 | 1.6 | 0.23 | 6.15 |
| Example 24 | faujasite | 200 | 0.8 | 1.3 | 0.62 |
| Example 25 | HCl | 170* | 0.6 | 3.0 | 0.2 |

* At 200° C. only acridine formation, reaction too fast to determine kinetics

Compared to the HCl catalyst, all zeolites suppressed the formation of acridine, indicating that the confined environment surrounding the acid site inhibits formation of bulky products.

### Isomerization of Mixtures of 2,2′-/2,4′-MDA to 4,4′-MDA

First, a catalyst (100 mg, see table 2), an industrial product feed comprising 2,2′- and 2,4′-MDA (500 mg) and 1.2 mL aniline were added to a GC vial. The GC vial was flushed with Ar and the vial was heated to 170° C. After the reaction, THE was added to dilute the reaction mixture. The obtained products were identified by GC-MS. For quantification, tetradecane was added as an external standard and analysis was done using GC. Rate constants were calculated according to Reference Example 2.

Further, the isomerization of an industrial product stream was tested with HCl (see FIG. 10) as catalyst and zeolite beta (see FIG. 11) as zeolite catalysts. Beta zeolite suppressed oligomerization and acridine formation and yielded an increased 4,4′-MDA.

### DESCRIPTION OF THE FIGURES

FIG. 1 shows a reaction scheme for the isomerization of 2,4′-MDA, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

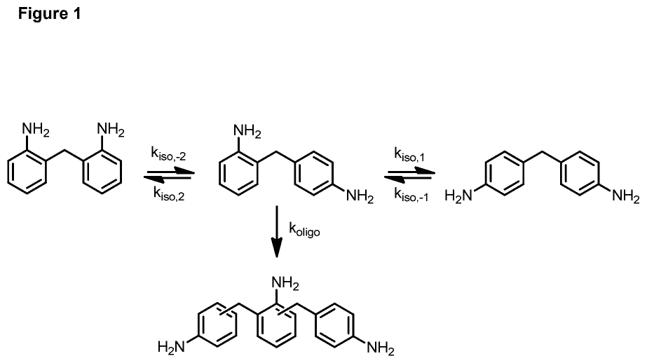

FIG. 2 shows the effect of aniline concentration on 2,4′-MDA isomerization, wherein the kinetic values are represented respectively by: ⋄ k_{iso,1} (2,4′→4,4′); ♦ k_{iso,2} (2,4′→2,2′) and ◯ k_{oligo}(MDA→oligomer).

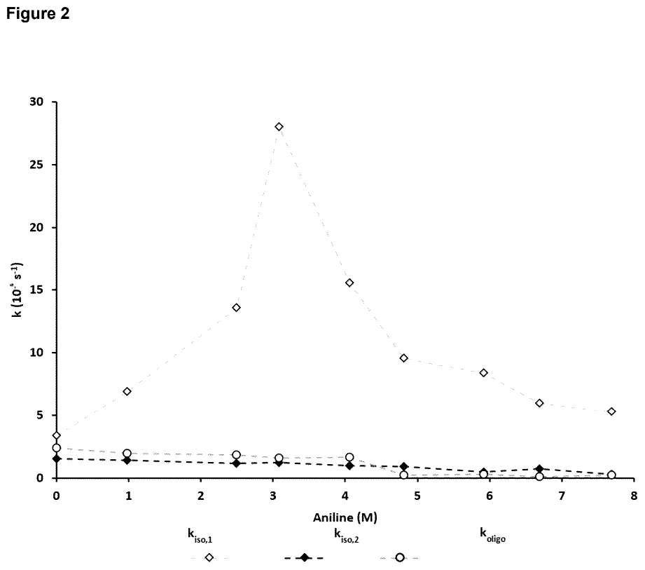

FIG. 3 shows the product distribution after the 2,4′-MDA isomerization using different catalysts.

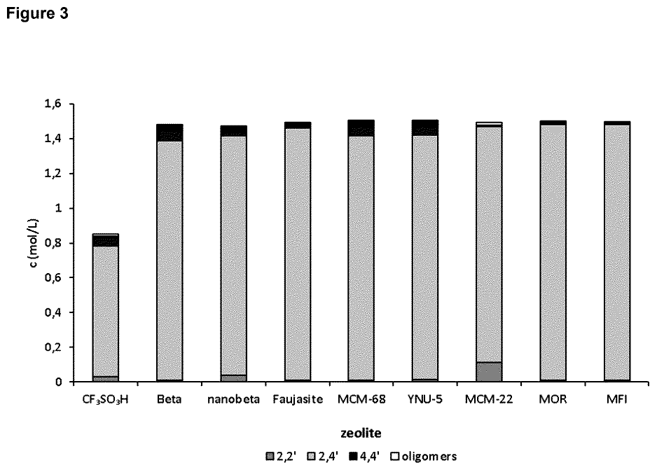

FIG. 4 shows the change in concentrations during 2,4′-MDA isomerization using CF₃SO₃H as catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

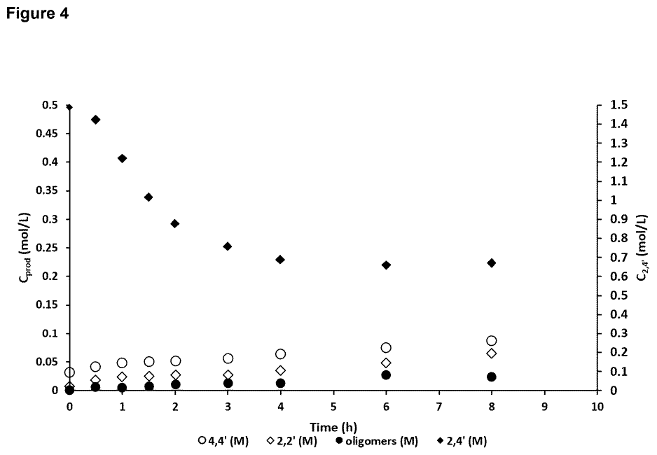

FIG. 5 shows the change in concentrations during 2,4′-MDA isomerization using a MFI zeolite catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

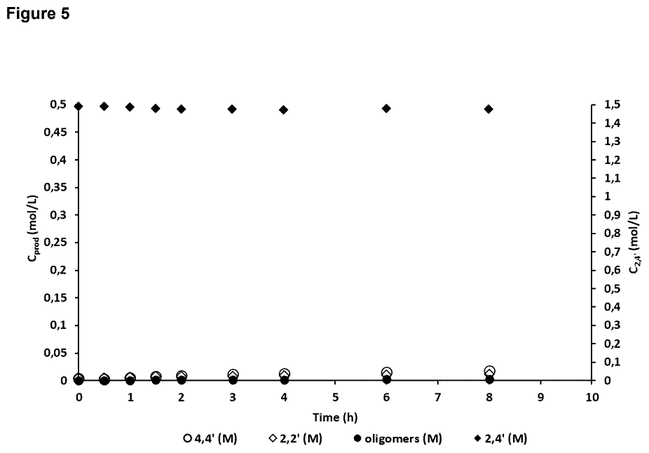

FIG. 6 shows the change in concentrations during 2,4′-MDA isomerization using HCl as catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

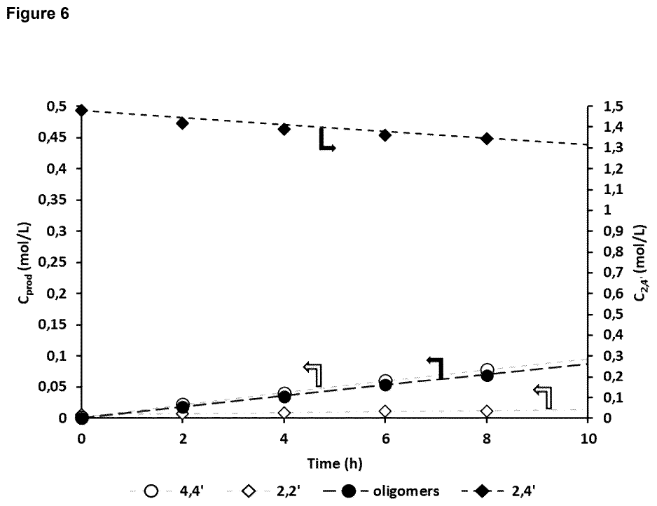

FIG. 7 shows the change in concentrations during 2,4′-MDA isomerization using a beta zeolite catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

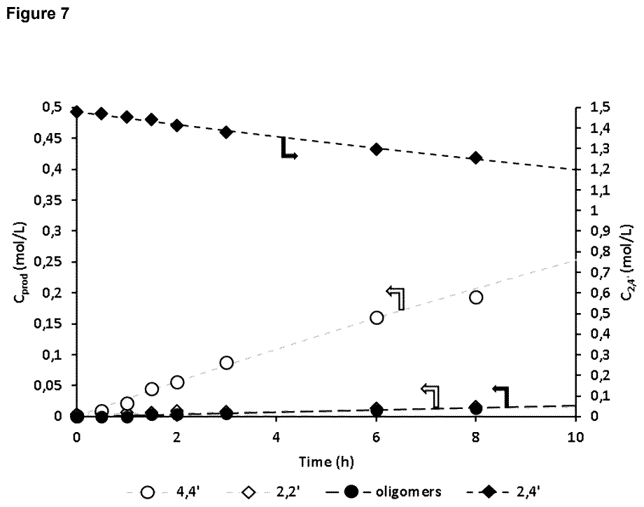

FIG. 8 shows the change in concentrations during 2,4′-MDA isomerization using a MCM-68 zeolite catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

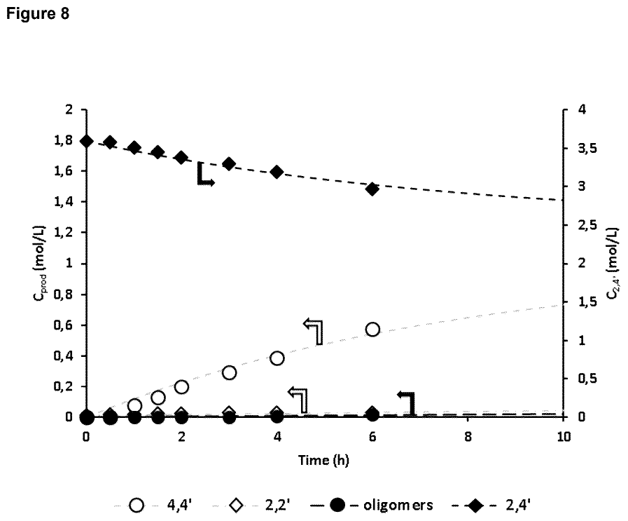

FIG. 9 shows the change in concentrations during 2,4′-MDA isomerization using a Y-UZM-35 zeolite catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer) and wherein an industrial waste stream is used.

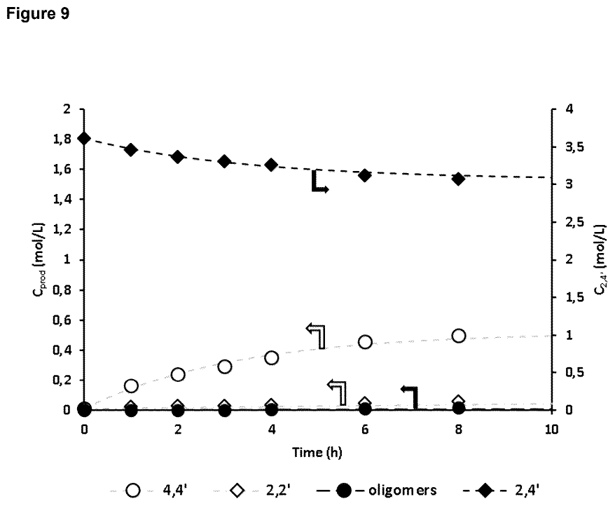

FIG. 10 shows the change in concentrations during the isomerization of an industrial product stream using HCl as catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer).

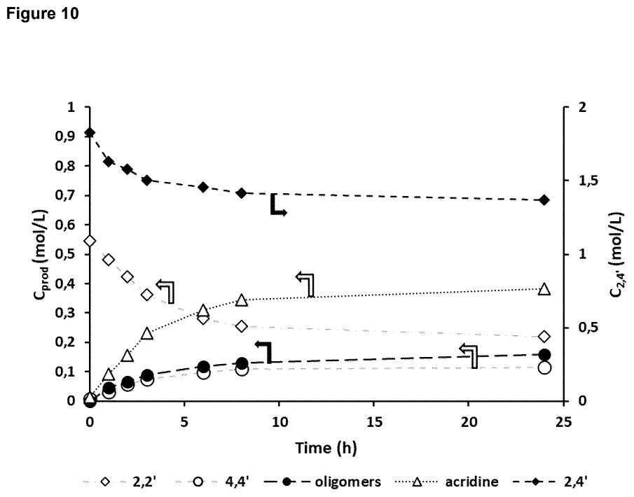

FIG. 11 shows the change in concentrations during the isomerization of an industrial product stream using a beta zeolite catalyst, wherein the kinetic values are represented respectively by: k_{iso,1} (2,4′→4,4′); k_{iso,-1}(4,4′→2,4′), k_{iso,2} (2,4′→2,2′), k_{iso,-2} (2,2′→2,4′) and k_{oligo}(MDA→oligomer) and wherein an industrial waste stream is used.

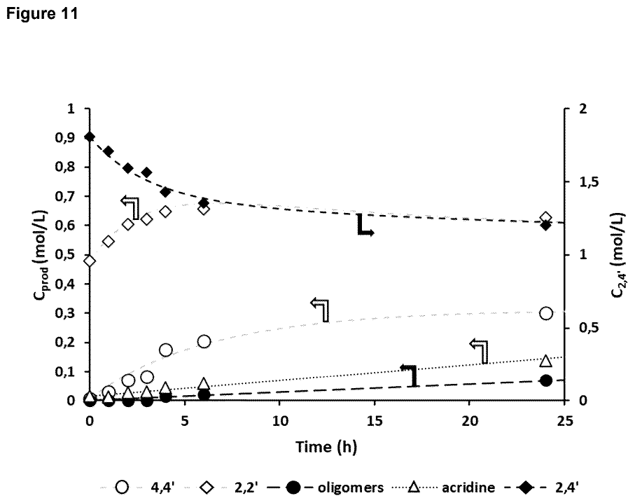

### CITED PRIOR ART DOCUMENTS

EP 0329367 Å2
WO 2021/116419 A
P. J. Whitman et al., ‘Protodealkylation of bis(aminophenyl)methanes’, Tetrahedron Letters, Vol. 27, pages 1887-1890

1.-16. (canceled)

17. A process for the isomerization of 2,4′-methylenedianiline and/or 2,2′-methylenedianiline to 4,4′-methylenedianiline, respectively, the process comprising
(1) preparing a feed F2 comprising 2,4′-methylenedianiline and/or 2,2′-methylenedianiline and optionally further comprising aniline;
(2) providing a catalyst C2 comprising a Bronsted acid;
(3) contacting the feed F2 prepared in (1) with the catalyst C2 provided in (2), obtaining a mixture M2 comprising 4,4′-methylenedianiline as a reaction product;
(4) optionally separating the 4,4′-methylenedianiline from the reaction product obtained in (3);
wherein the feed F2 in (1) comprises 5 wt.-% or less of formaldehyde.

18. The process according to claim 17, wherein the amount of 2,4′-methylenedianiline in the feed F2 in (1) ranges from 1 to 60 wt.-%.

19. The process according to claim 17, wherein the concentration of 4,4′-methylenedianiline in the mixture M2 obtained by (3) is greater than the concentration of 4,4′-methylenedianiline which may optionally be comprised in the feed F2 prepared in (1).

20. The process according to claim 17, wherein the molar ratio of 2,4′-methylenedianiline to 2,2′-methylenedianiline in the feed F2 in (1) is in the range of from 1:1 to 100:1.

21. The process according to claim 17, wherein the feed F2 in (1) comprises aniline, wherein the amount of aniline in the feed F2 in (1) ranges of from 45 to 90 wt.-%.

22. The process according to claim 17, wherein the feed F2 in (1) further comprises one or more of 4,4′-methylenedianiline, oligomers of 2,2′-methylenedianiline, oligomers of 2,4′-methylenedianiline, and oligomers of 4,4′-methylenedianiline.

23. The process according to claim 17, wherein the feed F2 in (1) is a product feed obtained from a process for the preparation of one or more of 4,4′-methylenedianiline, 2,2′-methylenedianiline, 2,4′-methylenedianiline, and oligomers thereof.

24. The process according to claim 17, wherein preparing the feed F2 in (1) comprises
(1.1) providing a feed Fla comprising aniline and a feed Fib comprising formaldehyde;
(1.2) contacting the feeds Fla and Fib provided in (1.1), obtaining a mixture M1b;
(1.3) optionally separating water from the mixture M1b obtained in (1.2), obtaining a Mixture M1c;
(1.4) providing a catalyst C1 comprising a Bronsted acid; and
(1.5) contacting the mixture M1b obtained in (1.2) or the mixture M1c obtained in (1.3) with the catalyst C1 provided in (1.4), obtaining a mixture M1a comprising 2,4′-methylenedianiline.

25. A process for the isomerization of 2,2′-methylenedianiline to 2,4′-methylenedianiline, the process comprising
(1′) preparing a feed F3 comprising 2,2′-methylenedianiline and optionally further comprising aniline;
(2′) providing a catalyst C3 comprising a Bronsted acid;
(3′) contacting the feed F3 prepared in (1′) with the catalyst C3 provided in (2′), obtaining a mixture M3 comprising 2,4′-methylenedianiline as a reaction product;
wherein the feed F3 in (1′) comprises 5 wt.-% or less of formaldehyde.

26. The process of claim 25, the process further comprising
(4′) providing a catalyst C2 comprising a Bronsted acid;
(5′) contacting the mixture M3 prepared in (3′) with the catalyst C2 provided in (4′), obtaining a mixture M4 comprising 4,4′-methylenedianiline as a reaction product;
(6′) optionally separating the 4,4′-methylenedianiline from the reaction product obtained in (5′).

27. The process according to claim 25, wherein independently from each other the Bronsted acid in (2′) and/or (4′) is a zeolite.

28. The process according to claim 25, wherein independently from each other the Bronsted acid in (2′) and/or (4′) is an inorganic acid.

29. The process according to claim 26, wherein the concentration of 4,4′-methylenedianiline in the mixture M4 obtained by (5′) is greater than the concentration of 4,4′-methylenedianiline which may optionally be comprised in the feed F3 prepared in (1′).

30. The process according to claim 25, wherein the feed F3 in (1′) comprises 2,2′-methylenedianiline and wherein the amount of 2,2′-methylenedianiline in the feed F3 ranges from 1 to 40 wt.-%.

31. The process according to claim 25, wherein the feed F3 in (1′) comprises aniline, wherein the amount of aniline in the feed F3 in (1′) ranges of from 45 to 90 wt.-%.

32. The process according to claim 25, wherein preparing the feed F3 in (1′) comprises
(1.1′) providing a feed Fla comprising aniline and a feed Fib comprising formaldehyde;
(1.2′) contacting the feeds Fla and Fib provided in (1.1′), obtaining a mixture Mib′;
(1.3′) optionally separating water from the mixture Mib′ obtained in (1.2′), obtaining a mixture Mic′;
(1.4′) providing a catalyst C1 comprising a Bronsted acid; and
(1.5′) contacting the mixture Mib′ obtained in (1.2′) or the mixture M1c′ obtained in (1.3′) with the catalyst C1 provided in (1.4′), obtaining a mixture M1a′ comprising 2,2′-methylenedianiline.
